(19) United States
(12) Patent Application Publication
(10) Pub. No.: US 2017/0333337 A1
(43) Pub. Date: Nov. 23, 2017

(54) COMPOSITION FOR SLOWING CELLULAR AGEING, ASSOCIATED FOOD SUPPLEMENT

(71) Applicant: Michel FREY, Paris (FR)

(72) Inventor: Michel FREY, Paris (FR)

(21) Appl. No.: 15/534,243

(22) PCT Filed: Dec. 7, 2015
(86) PCT No.: PCT/FR2015/053363

§ 371 Date: Jul. 5, 2017

(30) Foreign Application Priority Data
Dec. 9, 2014 (FR) 1462097

(51) Int. Cl. A61K 8/9789; A23L 33/10; A61K 8/67; A61K 8/63; A61K 8/60; A61K 8/55; A61K 8/19; A61K 8/11; A61K 8/04; A61K 8/02; A61K 8/9794; A23L 33/15; A23L 33/16; A23L 33/105; A61Q 19/08

(52) U.S. Cl. CPC A61K 8/9789; A61Q 19/08; A23L 33/10; A61K 8/673; A61K 8/63; A61K 8/606; A61K 8/55; A61K 8/19; A61K 8/11; A61K 8/042; A61K 8/0229; A61K 8/676; A61K 8/9794; A23L 33/15; A23L 33/16; A23L 33/105; A23V 2002/00

(57) ABSTRACT

The invention relates to a composition for slowing cellular ageing which comprises at least: S-Adenosyl-methionine; an extract of at least one adaptogenic plant selected among plants belonging to the Astragalus, Dioscorea, Schisandra, Panax, Eleuterococcus, Rosmarinus, Pfaffia, Morinda, Cordyceps or Lepidium genera; a vitamin selected among the B vitamins, vitamin C, or a mixture thereof; except for compositions comprising 7-keto-DHEA and an extract of one or more adaptogenic plants belonging to the Dioscorea or Lepidium genera. A liquid or solid food supplement can be characterised in that it comprises the composition. A dermatologically acceptable composition can be characterised in that it comprises at least 10 wt % of the composition.

### TECHNICAL FIELD OF THE INVENTION

The invention relates to the field of compositions intended for slowing signs of aging, in particular in a human being. Such a composition can be used as a food supplement or as a composition for topical application such as a cosmetic and/or dermatological and/or pharmaceutical cream or gel.

### PRIOR ART

It is accepted that, with age, various functions of the body deteriorate. There then may appear pathological and/or physiological signs related to aging, such as nerve degeneration characterised by the appearance of neurodegenerative illnesses such as Alzheimer's, Parkinson's, Huntington's chorea or motor neurone disease; decline in cognitive functions such as loss of memory or depression; osteoporosis; liver and/or digestive functional impairments; joint problems of cartilaginous or inflammatory origin such as rheumatoid arthritis; etc.

Given that life expectation continues to increase, it is now clear, in collective awareness, that in the future more and more people will be affected by such signs.

It has therefore become a major challenge to prevent the appearance of such signs and to slow the progression of aging.

There therefore exists a need to find compounds or compositions having the ability, in general terms, to restore the various functions of the human body, combatting and slowing the effects of aging.

### DISCLOSURE OF THE INVENTION

The invention aims to propose a composition able to slow cell aging, comprising at least:
S-Adenosyl-methionine,
an adaptogenic-plant extract,
a vitamin.

Advantageously, the composition for slowing cell aging comprising at least:
S-Adenosyl-methionine
an extract of at least one adaptogenic plant chosen from plants belonging to the Astragalus, Dioscorea, Schisandra, Panax, Eleuterococcus, Dioscorea, Rosmarinus, Pfaffia, Morinda, Cordyceps or Lepidium genera,
a vitamin chosen from vitamins in the B group, vitamin C, or a mixture thereof; excluding compounds comprising 7-keto-DHEA and an extract of one or more adaptogenic plants belonging to the Dioscorea or Lepidium genera.

Surprisingly the applicant discovered a synergy effect between the various compounds of the composition according to the invention making it possible to effectively and non-specifically combat stress whatever the origin thereof, as well as aging signs.

Interestingly, said adaptogenic-plant extract comprises: saponins chosen from type I to VII astragalosides, preferably of type I, II, II and/or IV, cycloastragenol. Thus the adaptogenic plant is chosen from the plants belonging to the Astragalus genus.

Thus S-Adenosyl-methionine (SAM) is a metabolite involved as a coenzyme in the activity of DNA or of histone methyltransferases. SAM supplementation prevents hypomethylation or hypermethylation of DNA having part responsibility in cardiovascular illnesses, liver ailments, depression and premature aging and in the development of cancer through various mechanisms.

In addition SAM is involved in the synthesis of epinephrin (adrenalin) by nucleophilic substitution reaction with norepinephrin; epinephrin is a hormone essential to the management of stress by our organism.

“Adaptogenic plant” means any plant increasing the ability of our body to adapt generally, that is to say non-specifically, to various stresses, whatever the origins thereof. Such a plant exerts a non-specific normalising action on numerous organs or physiological functions. The concept of “adaptogenic plant” exists in Chinese traditional medicine under the concept of “superior tonics”, which regularise various functions and increase energy, promoting health globally without treating specific illnesses. The group of adaptogenic plants comprising in particular plants belonging to the following genera: Astragalus, Dioscorea, Withania, Ginkgo, Schisandra, Panax, Eleuterococcus, Rhodiola, Schisandra, Schisandra, Lepidium, Rosmarinus, Pfaffia, Morinda, Cordyceps, Lepidium. For implementation of the invention, plants belonging to the Astragalus and/or Dioscorea and/or Lepidium genus will preferably be chosen.

Such plants, in particular Astragalus membranus (or astragal) or Dioscorea villosa or opposita or Lepidium meyenii (or maca) have the effect of restoring and normalising the various organs and functions of our organism, in particular by increasing the number of stem cells in the bone marrow and lymphatic tissue. These plants reactivate the immune cells at rest, by stimulating macrophages and promoting the regeneration of T and NK lymphocytes; inhibit lipid peroxidation in particular in the myocardium; and stimulate telomerase, thus extending the shortest telomeres, and protect DNA from cell aging.

“A vitamin” means all the vitamins: A, B1, B2, B3, B5, B6, B8, B9, B12, C, D, E K1, K2, their respective precursors, or a mixture of vitamins and/or respective precursors.

Advantageously, said vitamin is a vitamin in the B group, in particular B6, B9 or B12, or a mixture thereof.

B6 promotes in particular the absorption of magnesium and the synthesis of serotonin (an essential neurotransmitter).

Vitamin B9 or folic acid has the property of also acting on the metabolism of amino acids and the synthesis of proteins; it participates in the renewal of immune cells, in the formation of red corpuscles and in the supply of oxygen needed by the organism.

Vitamin B12 is essential to the correct functioning of the central and peripheral nervous systems.

Vitamin C is a powerful antioxidant limiting oxidative stress.

Advantageously, said composition comprises a magnesium salt.

Advantageously, said adaptogenic-plant extract comprises:
saponins, in particular type I to VIII astragalosides, preferably of type I, II, III and/or IV,
cycloastragenol.

Saponins, astragalosides, cycloastragenol and particularly the IV astragalosides appear to be some of the molecules responsible for the properties of adaptogenic plants with flavonoids, such as rhamnocitrin and betaines.

According to another feature, said adaptogenic-plant extract is produced from Astragalus, in particular Astragalus membranus, and/or Dioscorea, in particular Dioscorea villosa or opposita, and/or Lepidium, in particular Lepidium meyenii.

This is because plants in the Astragalus and Dioscorea genera contain flavonoids, polysaccharides and triterpene glycosides, which have the effect of combatting or limiting cell aging, such as the IV astragalosides and cycloastragenol.

“Magnesium salt” means any salt comprising magnesium and being biologically tolerated by the human body. The following can be cited as an example: magnesium chloride, magnesium citrate, magnesium gluconate, magnesium carbonate, magnesium pidolate, magnesium lactate, magnesium aspartate, magnesium sulphate, magnesium hydroxide, magnesium oxide, magnesium bisglycinate and magnesium glycerophosphate. The choice of the type of magnesium salt can be made according to the elementary magnesium content or according to the bioavailability and solubility of the magnesium salt.

The effect of the magnesium is to reduce inflammation, oxidative stress and endothelial malfunctionings (protective layer in the arteries). It also helps to reduce the formation of blood clots, which give rise to infarcts and certain strokes, and to reduce arterial pressure.

Advantageously, said magnesium salt chosen for implementing the invention is magnesium oxide or magnesium glycerophosphate; preferably said magnesium salt is of marine origin.

According to another feature, said composition is in the form of oil in water or water in oil emulsions, an aqueous or oily gel, a solid or any other forms suitable for topical, oral or injectable administration.

“Topical administration” means any administration by local method, for example on the skin, an orifice or mucosa. Topical administration, as used here, comprises the cutaneous, auricular, nasal, vaginal, urethral and rectal routes. For this type of administration, said composition is preferably in the form of a gel or an emulsion, preferably having properties of resistance to oxidation by the incorporation in the composition of antioxidants such as extracts of acai berry, pecan nut, hazelnut, prune, mulberry, pomegranate, strawberry, kiwi fruit, date, fig, garlic, almond, broccoli, beetroot, aubergine or any other known molecule having the property of limiting oxidative stress.

Preferably, and when it is necessary to add an oil, an essential oil will preferably be chosen, for example essential oil of helichrysum italicum to promote microcirculation, essential oil of Aniba parviflora for firming skin tissues, essential oil of Citrus limon, for its blood-fluidising power, essential oil of wintergreen for its vasodilatory action, essential oil of Lavandula officinalis for its healing and fluidising action, essential oil of Mentha piperita for its antipruritic power, essential oil of ravinsara for its skin energising action, essential oil of Melaleuca alternifolia for its skin antibacterial activity or essential oil of Pelagonium asperum for its “anti-wrinkle” power.

For administration of the oral type, the composition may be in the form of a liquid such as a syrup or a drinkable suspension, or in the form of a solid such as a tablet, a pill or a capsule. Preferably, the solid form is acid-resistant, in order to ensure disintegration of the constituents for assimilation thereof by the body, at the intestinal and non-gastric level. With such administration, the composition may be in galenic forms ranging from 5 mg to 2000 mg.

Advantageously, said composition according to the invention comprises at least:
between 0.01% and 70% by weight S-Adenosyl-methionine,
between 0.01% and 45% by weight adaptogen-plant extract,
between 0.01% and 40% by weight magnesium salt,
between 0.01% and 25% by weight vitamins.

More particularly, when the form of administration is topical, the composition according to the invention may preferably contain at least 30% by weight S-Adenosyl-methionine, more preferentially at least 50% by weight S-Adenosyl-methionine.

Another subject matter of the invention is a composition in the form of a solid mixture able to be introduced in a capsule, said composition comprises:
45% by weight S-Adenosyl-methionine
18% by weight adaptogen-plant extract,
36% by weight magnesium salt,
1% by weight vitamins.

Another subject matter of the invention is a food supplement, liquid or solid, comprising a composition according to the invention.

Another subject matter of the invention is a dermatologically acceptable composition, comprising at least 10% by weight, in particular at least 30% by weight, more preferentially at least 80% by weight, of a composition according to the invention.

“Dermatologically acceptable composition” means all compositions intended to be administered topically and respectful of the physical and chemical integrity of the skin and mucosa, such as so-called cosmetic, dermatological or pharmaceutical compositions.

### DETAILED DESCRIPTION OF VARIOUS EMBODIMENTS OF THE INVENTION

Numerous combinations can be envisaged without departing from the scope of the invention; a person skilled in the art will choose one or the other according to economic, ergonomic, dimensional or other constraints that he will have to comply with.

### Composition According to the Invention in the Form of a Food Supplement:

1

One embodiment of the invention is a food supplement in the form of an acid-resistant gelatine capsule (DR-caps from Casugel) able to contain in solid form:
250 mg of SAM (Laboratoire Prophar-Angers),
100 mg of a 20% dry extract of Astragalus membranaceus (Laboratoire Prophar-Angers),
200 mg of magnesium oxide of marine origin (Laboratoire Prophar-Angers),
4 mg of vitamin B6 representing 300% of the recommended daily intake (RDI) (Laboratoire Prophar-Angers),
800 μg of vitamin B12 (Laboratoire Prophar-Angers).

### Composition According to the Invention in the Form of a Food Supplement:

2

Another example embodiment of a food supplement in the form of an acid-resistant gelatine capsule according to the invention is as follows:
250 mg of SAM (Laboratoire Prophar-Angers),
200 mg of Dioscorea opposita in 20% dry extract (Laboratoire Prophar-Angers),
150 mg of magnesium oxide of marine origin (Laboratoire Prophar-Angers),
800 μg of vitamin B9, representing 200% of the recommended daily intake (RDI) (Laboratoire Prophar-Angers),
600 μg of vitamin B12 (Laboratoire Prophar Angers).

### Composition According to the Invention in the Form of a Food Supplement:

3

Another example of an embodiment of a food supplement in the form of an acid-resistant gelatine capsule according to the invention is as follows:
250 mg of SAM (Laboratoire Prophar-Angers),
200 mg of a 20% dry extract of Astragalus membranaceus (Laboratoire Prophar-Angers),
200 mg of Dioscorea villosa in a 20% dry extract (Laboratoire Prophar-Angers),
300 mg of magnesium oxide of marine origin (Laboratoire Prophar-Angers),
2 mg of vitamin B6 representing 200% of the recommended daily intake (RDI) (Laboratoire Prophar-Angers),
400 μg of vitamin B12 (Laboratoire Prophar-Angers).

### Composition According to the Invention in the Form of a Food Supplement:

4

Another example of an embodiment of a food supplement in the form of an acid-resistant gelatine capsule according to the invention is as follows:
250 mg of SAM (Laboratoire Prophar-Angers),
100 mg of a 20% dry extract of Astragalus membranaceus (Laboratoire Prophar-Angers),
100 mg of Dioscorea villosa in a 20% dry extract (Laboratoire Prophar-Angers),
300 mg of Lepidium meyenii in a 20% dry extract (Laboratoire Prophar-Angers),
300 mg of magnesium glycerophosphate of marine origin (Laboratoire Prophar-Angers),
2 mg of vitamin B6, representing 200% of the recommended daily intake (RDI) (Laboratoire Prophar-Angers),
500 μg of vitamin B9 (Laboratoire Prophar-Angers),
400 pg of vitamin B12 (Laboratoire Prophar-Angers).

### Composition According to the Invention in the Form of a Dermatologically Acceptable Composition

Another embodiment of the invention is a dermatologically acceptable composition in the form of a gel or emulsion, comprising at least 10% by weight, in particular at least 30% by weight, more preferentially at least 80% by weight, of a composition A according to the invention.

Preferentially, when a vehicle of the gel or oil type is used, the adaptogenic-plant extract used is then a hydroglycerin extract of a part of the plant such as the leaves, roots or stem, or a hydroglycerin extract of the entire plant.

For example, a dermatologically acceptable composition for cosmetic purposes comprises:
10% to 90% by total weight, for example 20% by total weight, of a vehicle of the non-alcoholic solid gel type, in the form of a bar or stick, and
10% to 90% by total weight, for example 80% by total weight, of a composition A according to the invention.

Said vehicle of the non-alcoholic solid cosmetic gel type, in the form of a bar or stick, comprises for example:
65% to 99% by weight of the vehicle of a polyol, chosen from glycerol, polyglycerol, polyethylene glycol, propylene glycol, sorbitol, alcohol ethers or a mixture of these,
0.1% to 5% by weight of a dibenzylidene-ose,
0.1% to 5% by weight of a hardening agent of the sulfosuccinate type, for example a silicone succinate at a concentration of between 1% and 1% by weight,
0.5% to 40% by weight water.

Said composition A according to the invention may comprise for example:
55% by weight S-Adenosyl-methionine,
35% by weight hydroglycerin extract of Dioscorea opposita and/or villosa,
10% by weight vitamin C or natural ascorbic acid, extract of acerola.

Another example of composition A is as follows:
40% by weight S-Adenosyl-methionine,
35% by weight hydroglycerin extract of Dioscorea villosa,
25% by weight vitamin C or natural ascorbic acid, extract of acerola.

In addition, in such dermatologically acceptable compositions, it is possible to incorporate active agents, or additional molecules of interest, such as perfumes, fragrances, preservatives, antioxidants, hydrating agents, anti-inflammatory agents, UV filters, as well as dyes, in a proportion of between 0.1% and 40% by total weight of said dermatologically acceptable composition. These active agents or additional molecules of interest neither alter nor affect the required properties of said dermatologically acceptable composition.

Such compositions according to the invention can be used in a protocol intended to combat or slow down the pathological or physiological signs of chronological and/or induced aging, such as the appearance of wrinkles, lines, loss of skin firmness, loss of skin elasticity, actinic keratosis, actinic moles, nervous degeneration characterised by the appearance of neurodegenerative illnesses such as Alzheimer's, Parkinson's, Huntington's Chorea or motor neurone disease, decline in cognitive functions, such as memory loss, depression, or signs of aging associated with osteoporosis; hepatic and/or digestive functional impairment; joint problems of cartilaginous or inflammatory origin such as rheumatoid polis arthritis; chronic inflammatory problems and any type of impairment or disease related to premature aging.

1. Composition for slowing cell aging comprising at least:
S-Adenosyl-methionine
an extract of at least one adaptogenic plant chosen from plants belonging to the Astragalus, Dioscorea, or Lepidium genera,
a vitamin chosen from vitamins in the B group, vitamin C, or a mixture thereof; excluding compounds comprising 7-keto-DHEA and an extract of one or more adaptogenic plants belonging to the Dioscorea or Lepidium genera.

2. Composition according to claim 1, characterised in that said adaptogenic-plant extract comprises:
saponins chosen from type I to VIII astragalosides, preferably of type I, II, III and/or IV,
cycloastragenol.

3. (canceled)

4. Composition according to claim 1, characterised in that said adaptogenic plant or plants is or are chosen from the plants belonging to the Astragalus genus.

5. Composition according to claim 1, characterised in that the vitamin in the B group is chosen from vitamins B6, B9, B12 or a mixture thereof

6. Composition according to claim 1, characterised in that said composition also comprises a magnesium salt.

7. Composition according to claim 1, characterised in that it comprises at least:
between 0.01% and 70% by weight S-Adenosyl-methionine,
between 0.01% and 45% by weight adaptogen-plant extract,
between 0.01% and 40% by weight magnesium salt, between 0.01% and 25% by weight vitamins.

8. Composition according to claim 1, characterised in that said composition is in the form of an oil in water or water in oil emulsions, an aqueous or oily gel, a solid or any other form suitable for topical, oral or injectable administration.

9. Composition according to claim 1, characterised in that, when said composition is in the form of a solid mixture able to be introduced in a capsule, said composition comprises:
45% by weight S-Adenosyl-methionine
18% by weight adaptogen-plant extract,
35% by weight magnesium salt,
1% by weight vitamins.

10. Liquid or solid food supplement, characterised in that it comprises a composition according to claim 1.

11. Dermatologically acceptable composition, comprising at least 80% by weigh of a composition according to claim 1.
